(19) United States
(12) Patent Application Publication
(10) Pub. No.: US 2005/0107752 A1
(43) Pub. Date: May 19, 2005

(54) Novel function, douching mode, is built into the hand-held or wall-mounted showerheads for women's personal hygiene

(76) Inventors: Ying Su, Lilburn, GA (US); Dianyi Yu, Lilburn, GA (US)

Correspondence Address: Dianyi Yu, 222 Black Birch Pass, Lilburn, GA, 30047, US

(21) Appl. No.: 10/715,040

(22) Filed: Nov. 17, 2003

(51) Int. Cl. A61M 31/00

(52) U.S. Cl. 604/275

(57) ABSTRACT

The novel idea of the present invention is to fabricate spiral configuration into the annular recessed cylindrical wall around the spray nozzle on the face plate of certain kinds of currently marketed hand-held and wall-mounted showerheads to allow a coupling feature in place for screw-on connection by our specially designed disposable vaginal douche nozzle. The showerhead water flow path for the douche nozzle shares the same water flow path with the spray nozzle. User can access the douche mode by selecting the existing spray mode of the spray nozzle wherein it has a coupling structure attachable to our douche nozzle. The spray pattern of the spray nozzle stays unaffected when our douche nozzle is not attached. In accordance with the present invention, adequate and efficient vaginal deodorization may be accomplished by douching with only normal household water that ensures minimal alteration of vaginal pH while rinsing out the vaginal canal.

### REFERENCES CITED (REFERENCED BY)

| | | | |
|---|---|---|---|
| 3968797 | July 1976 | Packer et al. | 604/215 |
| 3998390 | December 1976 | Peterson et al. | 239/394 |
| 4302186 | November 1981 | Cammack et al. | 433/80 |
| 5316216 | May 1994 | Cammack et al. | 239/71 |
| 5380300 | Jane 1995 | Pritchard et al. | 604/275 |
| 5476225 | December 1995 | Chan | 239/449 |
| 6589216 | July 2003 | Abbott et al. | 604/279 |
| 6626875 | September 2003 | Arzonico | 604/279 |

### OTHER REFERENCES

Douching—www.4women.gov—The National Women's Health Information Center, U.S. Department of Health and Human Services Office on Women's Health November 2002.

### BACKGROUND OF THE INVENTION

1.Field of the Invention

The present invention relates to showerheads and vaginal douche nozzles and, particularly, to the showerhead providing a novel function, douche mode, for women's personal hygiene through connecting a built-in coupling structure around spray nozzle on the face plate of hand-held or wall-mounted showerheads with a disposable vaginal douche nozzle. The method of douching presented in this invention provides a much easier, more convenient, more affordable, more reliable, more private, more enjoyable, and finally, more adequate and efficient way for women to rinse out the vaginal canal than any douching methods in prior art.

2. Description of the Prior Art

Douching is a practice that is thought to have been around since ancient times. Reasons that women have given for using douches include to: rinse away blood after a menstrual period; clean the vagina after sex to avoid sexually transmitted diseases (STDs), wash away semen to prevent pregnancy; and reduce odors. According to a report from the National Women's Health Information Center, U.S. Department of Health and Human Services, douching is a common practice among women in the United States—37% of American women between the ages of 15 to 44 douche regularly. Of these women, about half douche on a weekly basis. Although conventional vaginal douches may undesirably alter normal vaginal environments, douching has been found to benefit vaginal environments that are already undesirably altered or disturbed.

Vaginal Douching Device

Various douching devices have been developed for personal administration of a douche solution into the vagina. These devices are usually of a simple construction having a short stiff douche nozzle, sometimes curved along its length, for inserting along the vagina and a squeezable container for holding a quantity of douche solution and connectable to the nozzle. Manual squeezing of the container forces the solution to flow along the nozzle into the vagina. The terminal end of the nozzle is provided with a number of holes so that the solution is generally spray discharged. Users use household water or buy the douching solutions at drug and grocery stores. However, there are some obvious problems with these conventional douching devices. First, the commercial douching solutions may contain substances that cause irritation in some users and/or tend to alter the normal pH or chemical balance of the vaginal canal. Second, these douching solutions need advanced purchase and are costly if users douche frequently and regularly. Third, even though the normal household water is used as a douching fluid only, the conventional vaginal douche is not convenient when users need an adequate douching. The reason for that is that a squeezable container holds only a small quantity of douche solution (generally 6 oz) and users may have to refill the container with water and repeat the douching steps several times, which is obviously a frustrated and inconvenient operation, in order to obtain a relatively good rinse. Another drawback associated with conventional douching is that when the solution is discharged into vagina by manual squeezing, the pressure of fluid stream is not well under control so that the fluid discharge is erratic and the rinse through the vaginal canal is uneven, which may result in a poor quality of douching. Therefore, it has been found that in general these conventional douching devices provide only a limited douching effect.

Although there have been many inventions related to a vaginal douche device that attempted to solve these aforementioned problems, none of the inventions in prior art have become sufficiently easy, convenient, reliable, private, and visually enjoyable to become a popular product. Here are some examples: For irrigating and cleansing the vaginal canal, various forms of apparatus designed for attachment to a showerhead have been suggested as is apparent from U.S. Pat. Nos. 3,461,870; 3,512,525; 3,817,247; 3,847,150; 4,601,709; 4,911,704; 5,241,714; and 6,626,875. By attachment to a showerhead, users can easily adjust the temperature, volume, and pressure of the discharged fluid to the user's comfort and the fluid after use conveniently disappears into the shower drain. The common downside, however, of these inventions is the attachment between a showerhead and its water supply pipe that does not provide sufficient privacy of douche apparatus and requires extra work for the installation of the attachment device. The attachment is also visually unacceptable to some users due to the less natural look of the showerhead with a protruding attachment object around. Different from aforementioned inventions, U.S. Pat. No. 5,102,387 disclosed a transportable douche attachment which is adapted to be removably secured to a showerhead. This attachment comprises a nozzle, a cone having its reduced end attached to the nozzle and its enlarged end to engage the outer perimeter of the face of a showerhead, and a elongated ring of thin flexible material attached at one end of the enlarged end of the showerhead and adapted to be gathered behind the showerhead and held in place by a velcro strap. A conventional douche nozzle is secured to the opposite end of the cone to enable douching to occur. The design of this device, however, is only feasible to the hand-held showerhead. For those wall-mounted showerheads that are used commonly at home, hotels, and the like, this device would not be operable. In addition, users may have problems in operation because the cone materials need to be held in place by a strap, which is not convenient for consumers. Moreover, the cone, if not disposable, must be kept clean for repeated use, which is another drawback that causes inconvenience to the users. U.S. Pat. No.6,156,017 disclosed a cleaning device that needs two joints to get the device connected with showerhead water supply line, which is not practical and convenient to most douche users.

Showerheads

As stated earlier in the field of the invention, the present invention relates to showerheads. There are many showerheads which can deliver sprays in a plurality of different patterns. In general, there are two representative approaches to providing means for selecting between spray modes. One approach is to provide a face plate with a plurality of alternative spray heads formed therein which are sequentially placed in front of a water delivery passageway as the face plate is rotated. An example of such a showerhead is found in U.S. Pat. No. 3,998,390. A few of the other U.S. patents relating to this type of showerheads are U.S. Pat. Nos. 4,043,511; 4,165,837; 4,221,338; 4,657,185; 4,903,897; 5,070,552; 5,215,258; and 5,385,532. Another approach is to form a face plate with all spray orifices located in concentric circular patterns. An internal device may be operated to direct the incoming water to any of the circular patterns. An example of such a showerhead is found in U.S. Pat. No. 3,801,019. A few of the other U.S. patents relating to this type of showerheads are U.S. Pat. Nos. 3,958,756; 4,187,986; 4,190,207; 4,204,646; 4,303,201; 4,398,669; 4,588,130; 4,598,866; 5,172,866; 5,201,468; 5,316,216; 5,398,872; and 5,476,225. It does not matter to the present invention what mechanism is set forth in the prior art of showerheads providing means for spray mode selection. The novel concept of the present invention is about making a coupling structure around spray nozzle on the face plate of hand-held and wall-mounted showerheads. None of the prior art showerheads has borne a coupling feature around any kind or set of spray nozzle on the face plate to allow a showerhead to be attachable with a douche nozzle or the like directly or indirectly. The present invention hence discloses a novel function, douche mode, which can be easily built into the existing showerhead art where it is applicable and feasible to adopt the present invention with least cost increase on manufacture for the great benefits of women's personal hygiene.

Vaginal Douche Nozzles

As stated earlier in the field of the invention, the present invention also relates to vaginal douche nozzle. There are a few kinds of douche nozzles found from prior art. The U.S. Pat. No. 3,968,797 disclosed a douche nozzle attached to a liquid container. This nozzle does not address the concern on the pressure and direction of fluid flow at apertures provided in the spaced-apart grooves on the nozzle body, which, however, is an important issue that must be taken into account in the present invention. Moreover, the base portion of the douche nozzle from U.S. Pat. No. 3,968,797 is specially designed for attaching the container instead of having a coupling engagement as described from our douche nozzle. U.S. Pat. No. 5,380,300 relates to a douche nozzle for use with a squeeze bottle filled with a liquid. This nozzle also does not address the pressure and direction of fluid flow at apertures provided in the spaced-apart grooves on the nozzle body. Due to the straight shaft of the nozzle body, when attaching with a hand-held showerhead in our case, it can cause discomfort for user to hold the handle of the showerhead in order to obey the somewhat diagonally-upward insertion of douche nozzle into the vaginal canal. Also the fluid after use exiting vagina canal may fall on the face plate of the hand-held showerhead, which is unhygienic practice that is avoided by using our douche nozzle from the present invention. U.S. Pat. No. 6,626,875 discloses a device including a plurality of removable and disposable nozzles, where the apertures for discharging water from the douche nozzle are situated on the surface of the nozzle body. Since the vaginal canal is normally collapsed or contracted, it has a tendency to clamp down on a vaginal douche nozzle inserted therein. The apertures on the exterior surface of nozzle body may become “sealed” during use, which in turn brings about a poor quality of douching. Although some of prior art douche nozzles comprise grooves guiding the drainage of douche fluid downward towards vaginal opening, there is a concern whether or not the grooves provide enough drainage space between the apertures and vaginal wall when the douching fluid flow constantly in the case where the douche nozzle is connected with a showerhead. Our specially designed douche nozzle has taken this concern into account also. There are still other patents related to the douche nozzles from prior art, such as, U.S. Pat. Nos. 4,167,186; 5,013,297; 5,695,481; 6,190,365; 6,235,008; and 6,589,216. However, none of the prior art douche nozzles may be adapted to the showerhead with douche mode presented here without further significant modifications to meet our criteria.

To sum up, the present invention is to overcome the aforementioned disadvantages of all vaginal douching methods from prior art. With the showerhead being able to simply attach with a douche nozzle (i.e. hand-held showerhead) or a flexible douche hose (i.e. wall-mounted showerhead) without any aid of attachment or coupling device, as presented from this invention, women will enjoy such an adequate and efficient douching as frequently as they want in a much easier, more convenient, more affordable, more reliable, more private, and more enjoyable way than any other douching methods disclosed in prior art.

### SUMMARY OF THE INVENTION

Prior to the present invention, there is no hand-held or wall-mounted showerhead that has a coupling feature around any kind or set of spray nozzle on the showerhead face plate to allow a showerhead attachable with a vaginal douche nozzle or the like directly or indirectly. The novel idea of the present invention is to fabricate spiral configuration into the annular recessed cylindrical wall around the spray nozzle on the face plate of certain kinds of currently marketed hand-held and wall-mounted showerheads to allow a coupling feature in place for screw-on connection by a disposable vaginal douche nozzle (FIGS. 1 & 2). The showerheads bearing the features of the said spray nozzle surrounded by an annular recessed cylindrical wall can be found from many prior art showerheads disclosed, for example, in the U.S. Pat. Nos. 3,998,390; 4,043,511; 4,165,837; 4,221,338; 4,657,185; 4,903,897; 5,070,552; 5,215,258; and 5,385,532. The showerhead water flow path for the douche nozzle shares the same water flow path with the spray nozzle. In another words, user can access the douche mode by way of selecting the existing spray mode of the spray nozzle where it now has a coupling structure attachable to our douche nozzle. The spray pattern of the spray nozzle stays unaffected when a douche nozzle is not screwed onto the coupling structure on the showerhead face plate. The douche mode on the showerhead therefore is created by the present invention. The manufacturing cost for adding the spiral configuration into the annular recessed cylindrical wall around the said spray nozzle is believed to be reasonably inexpensive. This new douche feature on the showerhead, however, will bring a great deal of health benefits, convenience and joy to women.

In accordance with the present invention, effective vaginal deodorization may be accomplished by douching with only normal household water being used as the douching fluid. The use of water alone as the douching fluid ensures minimal alteration of vaginal pH while rinsing out the vaginal canal. The faucet as the water source for showerhead has a built-in valve, operable via a knob or knobs, for selectively controlling water pressure, volume, and temperature of douching water flow supplied to the vaginal douche nozzle. A preferred pressure, volume, and temperature of the water flow from a nozzle are easily set by the user through operating the knob or knobs of the faucet controlling the water supply of the showerhead. The douching water enters and exits vaginal canal easily by using our specially designed douche nozzle (FIG. 3) attachable to a showerhead with douche mode.

Referring back to the problems associated with the prior art vaginal douching devices discussed earlier, the advantages of the method of douching presented in this invention are obvious. Unlike the conventional douche devices that usually have a douche nozzle connected with a squeezable bottle, the present invention promotes more frequent vaginal douching as a preventive for unbalanced vaginal environments, particularly post-coital douching, since the normal household water and a showerhead having douche mode can be so handy for use on demand and eliminate the need for advanced planning and purchases for douching solutions. More importantly, this invention provides an adequate and efficient douching through a thorough and even vaginal rinse by normal household water with controlled water pressure, volume, and temperature. Also unlike those douche devices using an attachment device between a showerhead and its water supply pipe to allow the vaginal douching to occur, the present invention has its douche feature built into the hand-held and wall-mounted showerheads to provide more privacy of the douche apparatus and natural appearance of the showerhead, to avoid installation of any piece of attachment device around showerhead water supply pipe, and to offer women a great convenience on douching practice. In addition, the douche nozzle presented in this invention is a functional mate that has all the features required for this invention to function effectively and efficiently.

It is, therefore, a primary object of our present invention to overcome the disadvantages of prior vaginal douche art.

Another object of the present invention is to provide an easier, more convenient, more affordable, more reliable, more private, more enjoyable, and finally, more adequate and efficient way of douching for women to rinse out the vaginal canal than any douching methods in prior art.

A further object of the present invention is to provide a new and improved douching method that is acceptable and popular to the majority of women who douche on a regular basis.

A still further object of the present invention is to promote more frequent vaginal douching as a preventive for unbalanced vaginal environments.

A related object of the present invention is to create a novel function, douche mode, into the existing hand-held and wall-mounted showerheads art for women's personal hygiene.

A further related object of the present invention is to provide a vaginal douche nozzle that serves as a functional mate that has all the features required for this invention to function effectively and efficiently.

Other objects and advantages of the present invention will become apparent from the following detailed description of the invention taken in conjunction with the accompanying drawings.

### BRIEF DESCRIPTION OF THE DRAWINGS

FIG. 1 is an exploded perspective view showing a built-in coupling feature in the spray nozzle on the face plate of a hand-held showerhead for screw-on connection with a disposable vaginal douche nozzle.

FIG. 2 is an enlarged view of the built-in coupling structure showing the spiral configuration structured onto the annular recessed cylindrical wall surrounding the spray nozzle on the showerhead face plate depicted in FIG. 1.

FIG. 3 is an exploded perspective view showing the same built-in coupling feature in the spray nozzle on the face plate as the one shown in FIG. 1 but for a wall-mounted showerhead with a flexible douche hose that connects on another end a disposable vaginal douche nozzle.

FIG. 4 is a view of the longitudinal section crossing bilaterally symmetric grooves of the douche nozzle.

FIG. 5 is a view of the longitudinal section crossing bilaterally symmetric ridges of the douche nozzle.

FIG. 6 is a cross-sectional view of the joint end of the douche nozzle.

FIG. 7 is a cross-sectional view of the angled portion of the douche nozzle.

FIG. 8 is a view of a cross section of the upper body of the douche nozzle that missed the apertures within the grooves and bumps on the ridges.

FIG. 9 is a view of a cross section of the upper body of the douche nozzle that crossed the apertures in the grooves and bumps on the ridges.

FIG. 10 is an alternative version of a built-in coupling structure showing the external spiral configuration structured onto the barrel-shape spray nozzle on the showerhead face plate.

FIG. 11 is a cross-sectional view of snap-fit coupling components fitted together in operative position.

### DETAILED DESCRIPTION OF THE INVENTION

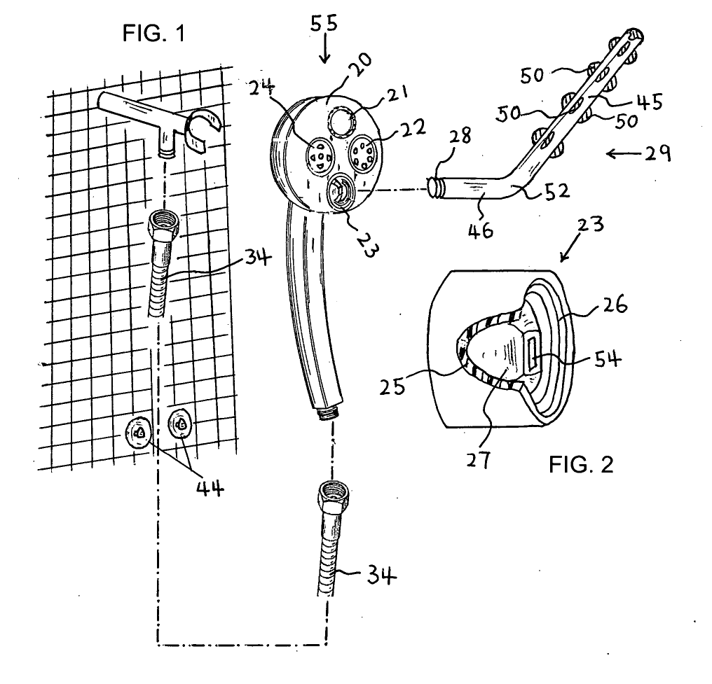

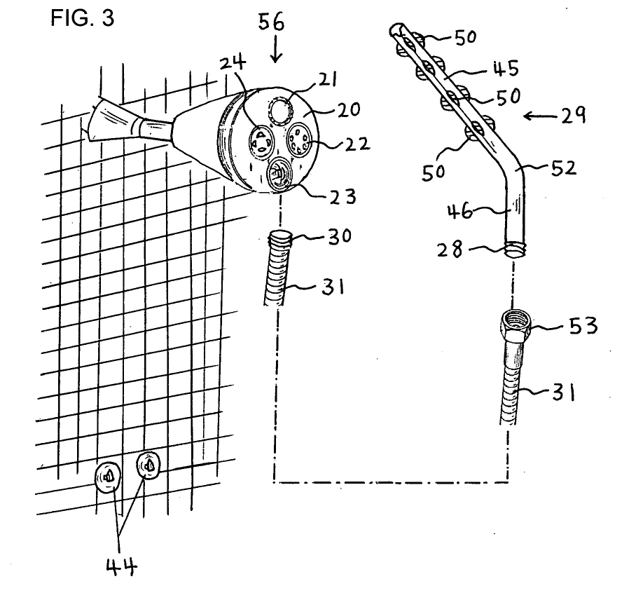

A novel function, douche mode, built into the hand-held showerhead 55 and wall-mounted showerhead 56 for women's personal hygiene according to the present invention is illustrated in FIGS. 1-3. The face plate 20 of either hand-held or wall-mounted showerheads has a few individual spray nozzle patterns 21-24. The individual spray nozzle 23 consists of a dam-shape spray nozzle 27 with a rectangular discharge orifice 54 and an annular recessed cylindrical wall 25. Such or similar type of individual spray nozzle structure has been found in many prior art showerheads that have been widely marketed for decades. The novel idea of the present invention is to make spiral configuration 26 into the annular recessed cylindrical wall 25 around the dam-shape spray nozzle 27 on the face plate 20 of either a hand-held showerhead 55 or a wall-mounted showerhead 56 to allow a coupling feature 26 in place for screw-on connection with the joint end 28 of a disposable vaginal douche nozzle 29 or the first end 30 of a flexible douche hose 31 which is attachable through its second end 53 to a douche nozzle joint end 28. The showerhead water flow path for the douche nozzle 29 shares the same water flow path with the spray nozzle 27. In another words, user can access the douche mode by way of selecting the existing spray mode of the spray nozzle 27 where it has a coupling structure 26 attachable to a douche nozzle 29. The spray pattern of the spray nozzle 27 stays unaffected when a douche nozzle 29 is not screwed onto the coupling structure 26 on the face plate 20. The douche mode on the showerhead therefore is created by the present invention.

In FIG. 1, the faucet 44 as the water source for showerhead has a built-in valve, operable via a knob or knobs, for selectively controlling the pressure, volume, and temperature of douching water supplied to the vaginal douche nozzle 29. The water flow from a douche nozzle 29 is easily set by users through operating the knob or knobs of the faucet 44 controlling the water supply of the showerhead.

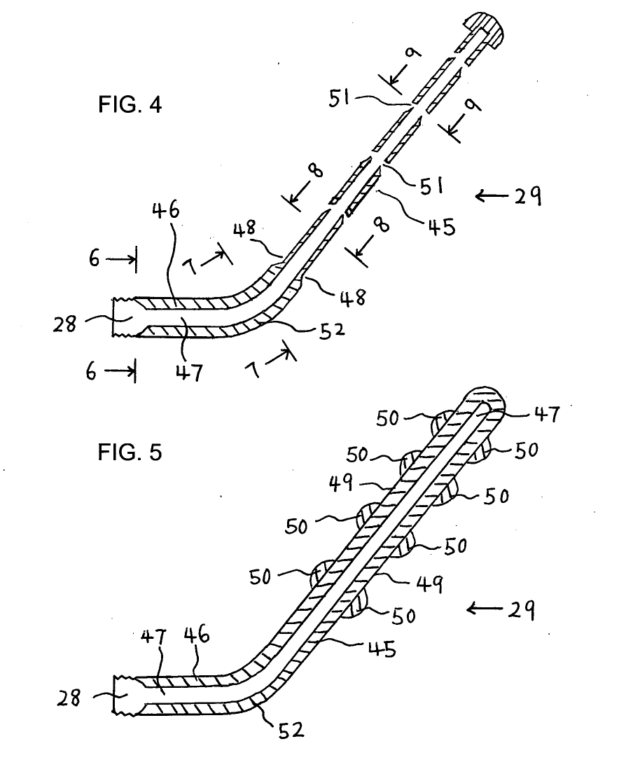

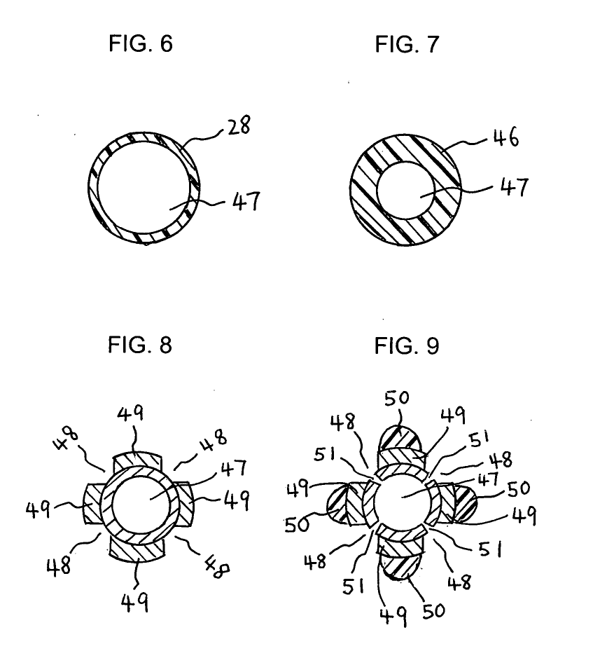

The douching water enters and exits vaginal canal easily by using our specially designed douche nozzle 29. Illustrated in the FIGS. 4 & 5, the douche nozzle 29 comprises a hollow tubular upper body 45, a hollow tubular lower body 46, and a joint end 28 having an externally threaded configuration. The nozzle 29 is hollow thereby forming a fluid-flow passageway 47. There are four recessed spaced-apart grooves 48 and accordingly four longitudinal ridges 49 with four spaced-apart hemispherical bumps 50 along longitudinal axis of each ridge 49 on the exterior surface of the nozzle upper body 45 (FIGS. 4, 5, 8 & 9). The grooves 48 and bumps 50 on the ridges 49 together prevent the vaginal canal from “sealing” apertures 51 nestled within the grooves 48 of a douche nozzle 29 so as to help maintain an efficient drainage of douching fluid out of vaginal canal. Also the unique feature of these bumps 50 may help clean out vaginal foul secretion, such as leukorrhea, adhered to the vaginal wall when user slightly moves the douche nozzle 29 up and down in the vagina. The douche nozzle 29 is curved with an obtuse angle 52 between the upper body 45 and lower body 46 in order to avoid both uncomfortable posture from holding the showerhead handle to adjust the insertion angle of a straight douche nozzle into the vagina and douching water after use falling from the vaginal canal on the face plate of the showerhead. The joint end 28 of a douche nozzle 29 has the largest diameter (FIG. 6) of the entire fluid-flow passageway 47. The diameter tapers quickly to the same regular size (FIG. 7) as the rest of water passageway 47 is in the douche nozzle body. A plurality of apertures 51 (FIGS. 4 & 9) are provided through the entire length of each groove 48 and communicate with the fluid passageway 47 in the nozzle upper body 45. There are a plurality of apertures 51 on the douche nozzle upper body 45 to allow water to exit the douche nozzle 29 at a relatively gentle pressure so as to avoid the possibility of excess water force to be introduced into the vaginal canal. The apertures 51 are located within the recessed grooves 48 in order to prevent the apertures 51 from becoming “sealed” by the contracted vaginal canal where it has a tendency to clamp down on a vaginal douche nozzle inserted therein. The apertures 51 are structured in diagonally-downward cone-shape having its reduced end towards the hollow fluid passageway 47 and its enlarged end open to the exterior surface of the douche nozzle upper body 45. This configuration of apertures can help reduce the pressure of water flow and control the water to exit the douche nozzle upper body 45 in a diagonally-downward direction such that the douching water is directed through the grooves 48 and towards the vaginal opening. The douche nozzle lower body 46 has a smooth exterior surface and basically serves as a connecting tunnel between the joint end 28 and the douche nozzle upper body 45. The joint end 28 has externally threaded configuration that is enabled to screw onto the spirally configured recessed cylindrical wall 26 around the spray nozzle 27 for a coupling engagement.

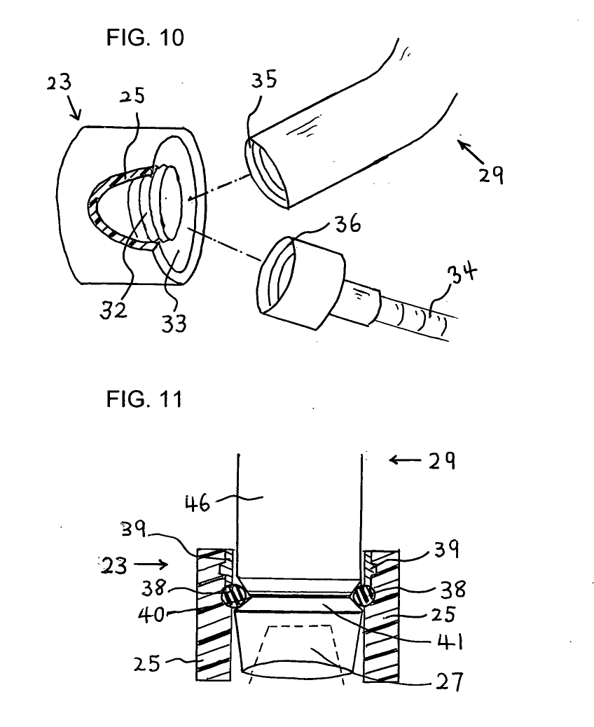

One alternative version, depicted in FIG. 10, of the coupling engagement between the spray nozzle 27 and the douche nozzle 29 or douche hose 31 is to make the dam-shape spray nozzle 27 into a barrel-shape spray nozzle 32. The outer clearance space 33 between the barrel-shape spray nozzle 32 and the annular recessed cylindrical wall 25 is used for a douche nozzle with an internally threaded joint end 35 or the connecting end 36 of a regular flexible shower hose 34 to twist on for a coupling engagement. Another alternative version, depicted in FIG. 11, of a coupling engagement is to have the joint end 41 of the douche nozzle 29 secured with a snap fit in the annular recessed cylindrical wall 25 by a floating O-ring 38. The latter is captivated in the bore of the annular recessed cylindrical wall 25 by a retaining sleeve 39 that defines a recess. The snap fit is effected by a groove 40 formed around the joint end 41 of the douche nozzle 29. Still, the water flow path for the douche nozzle 29 shares the same water flow path with the spray nozzle 27. The spray pattern of the spray nozzle 27 stays unaffected when a douche nozzle joint end 41 is not snapped into the spray nozzle 23 bearing a snap fit coupling feature described above on the face plate 20.

Alternative option to allow an angle between the upper body 45 and lower body 46 of the douche nozzle is to use flexible plastic at the portion of lower body 46 of the douche nozzle 29 so that the douche nozzle 29 can be curved to a certain angle at the douche nozzle lower body 46.

While the present invention has been described in connection with what is considered the most practical and preferred embodiment, it is understood that this invention is not limited to the disclosed embodiment but is intended to cover various arrangements included within the spirit and scope of the broadest interpretations and equivalent arrangements.

1. The present invention is about bringing a novel function, douche mode, into the hand-held and wall-mounted showerheads for women's personal hygiene by making spiral configuration into the annular recessed cylindrical wall around the spray nozzle on the face plate of hand-held and wall-mounted showerheads to allow a coupling feature in place for screw-on connection by a disposable vaginal douche nozzle or by a flexible douche hose attachable to a disposable vaginal douche nozzle, so that the showerhead water flow path for the disposable vaginal douche nozzle shares the same water flow path with the said spray nozzle and that the spray pattern of the said spray nozzle is unaffected when a disposable vaginal douche nozzle is not screwed onto the coupling structure on the said showerhead face plate, thus resulting in a douche mode created on the showerhead.

2. The showerhead douche mode as defined in claim 1 wherein the coupling engagement between the said spray nozzle and the douche nozzle or the said douche hose may alternatively comprise
a barrel-shape spray nozzle with externally threaded configuration;
an annular clearance space between the said externally threaded barrel-shape spray nozzle and the annular recessed cylindrical wall to allow a douche nozzle with an internally threaded joint end or a regular flexible shower hose to twist on for a coupling engagement, so that the showerhead water flow path for the said douche nozzle shares the same water flow path with the said barrel-shape spray nozzle and that the spray pattern of the said barrel-shape spray nozzle is unaffected when the said douche nozzle is not twisted onto the coupling structure on the showerhead face plate, thus resulting in a douche mode created on the showerhead.

3. The showerhead douche mode as defined in claim 1 wherein the coupling engagement between the said spray nozzle and the said douche nozzle or the said douche hose may alternatively consist of
a douche nozzle with the annular external groove formed around the joint end that can be secured with a snap fit in the annular recessed cylindrical wall by a floating O-ring;
the said floating O-ring that is captivated in the bore of the annular recessed cylindrical wall by a retaining sleeve that defines a recess;
the snap fit feature which is effected by the said groove formed around the joint end of the disposable douche nozzle, so that the showerhead water flow path for the disposable vaginal douche nozzle shares the same water flow path with the said spray nozzle and that the spray pattern of the said spray nozzle is unaffected when a disposable and removable vaginal douche nozzle is not snapped onto the coupling structure on the said showerhead face plate, thus resulting in a douche mode created on the showerhead.

4. The showerhead douche mode as defined in claim 1 wherein the said douche nozzle comprises
a hollow tubular upper body, a hollow tubular lower body, and a joint end;
a joint end that has at least three versions of coupling structures: externally threaded configuration (screw-on type), internally threaded configuration (twist-on type), or groove configuration formed around the joint end (snap-fit type);
four recessed spaced-apart grooves extending longitudinally on the exterior surface of the douche nozzle upper body;
four ridges with four spaced-apart hemispherical bumps along longitudinal axis of each ridge between the said recessed spaced-apart grooves on the exterior surface of the douche nozzle upper body;
a fixed or flexible obtuse angle between the douche nozzle upper body and douche nozzle lower body;
a plurality of apertures nestled within the said four recessed spaced-apart grooves on the douche nozzle upper body;
the said apertures structured in diagonally-downward cone-shape having its reduced end towards the hollow fluid passageway and its enlarged end open to the exterior surface of the douche nozzle upper body;
the douche nozzle lower body that has smooth exterior surface and basically serves as a connecting tunnel between the joint end and the douche nozzle upper body.

5. The showerhead douche mode as defined in claim 1 wherein the said douche hose comprises
a first end, a flexible hose, and a second end;
a first end that has an externally threaded configuration to be attachable to the said coupling feature in claim 1 in the showerhead face plate and a second end that connects with a disposable douche nozzle;
a first end that alternatively has an internally threaded configuration to be attachable to the said coupling feature in claim 2 in the showerhead face plate and a second end that connects with a disposable douche nozzle;
a first end that alternatively has an annular groove formed around the end, as the joint end of the douche nozzle in claim 3, which is secured but removable with a snap fit in the showerhead face plate and a second end that connects with a disposable douche nozzle.
